(19) United States
(12) Patent Application Publication
(10) Pub. No.: US 2024/0230657 A1
(43) Pub. Date: Jul. 11, 2024

(54) SIMPLIFIED QUANTITATIVE ANALYSIS USING SUBSTRATES WITH PRE-LOADED STANDARDS

(71) Applicants: UNIVERSITY OF FLORIDARESEARCH FOUNDATION, INCORPORATED, Gainesville, FL (US); Capitainer AB, Solna (SE)

(72) Inventors: Timothy J. GARRETT, Gainesville, FL (US); Donald CHACE, Gainesville, FL (US)

(21) Appl. No.: 18/409,738

(22) Filed: Jan. 10, 2024

Related U.S. Application Data
(60) Provisional application No. 63/438,082, filed on Jan. 10, 2023.

(51) Int. Cl. G01N 33/58; B01L 3/00; G01N 33/68

(52) U.S. Cl. CPC G01N 33/58; B01L 3/502715; G01N 33/6806; B01L 2300/0803

(57) ABSTRACT

Dried blood spots (DBS) collected on a substrate material are used in the quantitation of amino acids, acylcarnitines, organic acids and numerous other small molecules. One of their main application areas is newborn screening. In order to properly quantitate small molecules of target analytes in DBS, stable isotope labeled internal standards that are typically deuterium or carbon 13 labeled versions of the desired target analytes to be quantitated are pre-loaded onto the substrate before blood collection. For example, phenylalanine with 6 Carbon 13 atoms is used as a reference to quantitate phenylalanine.

### FIELD OF THE INVENTION

The present invention relates to a method and a microfluidic device for determining the quantity of target analytes such as metabolites including peptides, proteins, heavy metals, toxins, drugs and their metabolites or other molecules in bio-samples by using labeled internal standards, in particular pre-loaded stable isotope labeled internal standards onto a substrate that is to be loaded with a bio-sample such as for diagnosis of various metabolic disorders, including newborn screening, cancer, diabetes, cardiovascular disease, drug monitoring, and others.

### BACKGROUND

### Metabolomics

Metabolomics offers a promising answer to this diagnostic challenge. The Metabolome is entire set of metabolites in a given system while the lipidome is entire set of lipid features; both are the major molecular components of biological systems.^{1, 2} Metabolites and lipids are both the input and the output of cellular and physiological processes, and their levels are therefore exquisitely sensitive to a wide range of perturbations linked to disease, genetic modification, and environmental conditions.^{3, 4} Metabolomics is the accurate and precise measurement of metabolites in a given biological medium making metabolomics and lipidomics emerging approaches in many different areas; for example, cancer research^{5, 6}, diabetes⁷, gut microbiome^{8, 9}, and newborn screening^{10, 11}. In cancer, tumor cells predominantly reprogram metabolism in demand to satisfy three major functions: bioenergetics (central carbon metabolism), biosynthesis (the production of biomaterials such as nucleotides, amino acids, and lipids), and redox balance (chemistry of oxidation-reduction homeostasis status in metabolism).^{12, 13} These processes together support cancer cell maintenance, proliferation, tumor initiation, growth, and progression and are considered central dogma to cancer metabolism allowing cancer cells to adapt to changing cellular and physiological conditions in context to growth-related signal/stress. Findings from genomics, epigenomics, proteomics, and transcriptomics have tremendously expanded our understanding of cancer and have elucidated novel and potentially targetable tumor vulnerabilities.¹⁴ Altered metabolism is recognized as a cancer hallmark, offering invaluable opportunities for cancer diagnostics, prognostics, and therapeutics.¹⁵⁻¹⁸ Notably, there is growing clinical success of anti-metabolites for treating cancer attributed to the increased metabolic demand of cancer cells for nucleotide biosynthesis, glycolysis, TCA cycle, serine biosynthesis and folate cycle, methionine cycle, and fatty acid synthesis; however, their targets and associated pathways only target a few of the many possible metabolic dependencies altered to support cancer cell proliferation and tumor progression.^{12, 13, 19}

### Inborn Errors of Metabolism

Inborn errors of metabolism (IEMs) are a group of various genetic metabolic disorders, each of which is caused by an error in a single genetic code that results in insufficient or deficient enzyme activity required for intermediary metabolism.

Delay in treatment of metabolic disorders of IEM may lead to a variety of adverse outcomes, including moderate-to-severe morbidity such as neuropsychological dysfunction and mental retardation, and mortality due to impaired metabolism and consequently due to lack or accumulation of certain metabolic intermediates. Therefore, early diagnosis is important for timely correction of the symptoms with dietary or drug interventions before the clinical manifestations become evident in affected newborns. Each year, about 4 million of babies in the U.S. are routinely screened for potential metabolic disorders through newborn screening.

Newborn screening is a set of tests for the recognition and management of conditions that can affect a child's long-term health or survival at their earliest in order to prevent severe clinical symptoms, disabilities and death associated. Newborn screening for metabolic disorders of IEMs is performed by collecting a few drops of blood of a baby onto a special type of substrate such as a cotton disk. The blood on the substrate is dried and then sent to the State Health Department. The dried blood spot (DBS) is extracted and analyzed by the lab for the quantitation of numerous metabolites and small molecules such as amino acids, free carnitine, acyl-carnitines and organic acids to identify babies who are at high risk to have a medical condition.

For the quantitation of metabolites and other small molecules in bio-samples, which are the target analytes of IEM diagnosis with newborn screening, internal standards (IS) are used as reference. Usually, internal standards are stable isotope labeled versions of the desired target analytes, and the stable isotopes are typically deuterium or carbon 13. For example, to quantify phenylalanine in a test sample such as blood, phenylalanine labeled with 6 carbon 13 atoms is used.

Quantitation of target analytes by adding internal standards to dried blood spot (DBS) samples can be complicated. Currently, internal standards are added when the extraction solvent is added to the sample (blood)-loaded substrate. However, this use of the internal standards does not reflect the extraction efficiency of the target analytes from the substrate nor does it reflect any potential degradation that could occur to the sample prior to analysis. In addition, the loaded sample such as blood can vary in size, volume and hematocrit, making precision quantitation difficult.

### SUMMARY OF INVENTION

An object of the present invention is to provide an improved method and device for overcoming all or some of the disadvantages and problems described above in connection with the state of the art.

In this disclosure, the feasibility of the new approach of using-pre-loaded internal standards as a reference was demonstrated for the quantitation of target molecules. Based on the discoveries reported herein, one embodiment of the invention pertains to a method for determining the quantity of at least one target molecule in a fluid test sample. The method involves delivering a predetermined volume of the fluid test sample to a substrate pre-loaded with a predetermined amount of internal standard molecules such that the predetermined volume of the fluid test sample contacts the internal standard molecules. The fluid test sample may be dried after delivery to the substrate. The substrate or a portion thereof with the fluid test sample is incubated in an extraction solvent so as to produce a supernatant that comprises the at least one target molecule and the internal standard molecules. The method also involves detecting the at least one target molecule and the internal standard molecules in the supernatant; and quantifying the amount of the at least one target molecule in the fluid test sample based on the amount the at least one target molecule and the amount of the internal standard molecules detected in the supernatant. In a specific embodiment, and for the extraction experiments described below, the substrate (e.g. cotton disk) is part of a volumetric microsampling devices such as a quantitative dried blood sample (qDBS) card

Embodiments also pertain to method and/or a microfluidic device for determining the quantity of at least one target molecule in a test sample by using internal standard molecules of known amount. The embodiments may be used to screen or diagnose a variety of medical conditions, including diagnosing metabolic disorders. In typical embodiments, the internal standard is a stable isotope labeled internal standard molecule corresponding to the target molecule labeled with at least one stable isotope, wherein the stable isotope for labeling the internal standard molecule is selected from the group consisting of ²H, ¹³C, ¹⁵N, ¹⁸O, ³⁴S, or any combinations thereof, and, optionally, wherein the stable isotope labeled internal standard molecule may be in an amount range of 1 fmol to 5 mmol. Those skilled in the art will appreciate that, depending on the target molecule, unlabeled internal standards may be implemented. It is contemplated that unlabeled internal standards would be particularly useful in analyzation methods apart from mass spectroscopy (e.g., ELIZA, chromatography and the like).

In addition, the test sample for which this method and microfluidic device can be used is selected from biofluid such as excreted fluid (such as urine or sweat), secreted fluid (such as saliva, tears, breast milk or bile), fluid obtained within a subject (such as blood, plasma, serum or cerebrospinal fluid), or fluid generated as a result of a pathological process (such as blister or cyst fluid), food sample, plant sample, environmental sample or the like.

In a specific embodiment, disclosed is a method that comprises the steps of
(i) loading a known amount of solution comprising at least one of stable isotope labeled internal standard molecules onto a substrate,
(ii) drying the substrate for a predetermined time, e.g. 2-48 hrs,
(iii) adding a known volume of the fluid test sample onto the substrate preloaded with at least one of stable isotope labeled internal standard molecules;
(iv) drying the substrate sufficiently,
(v) putting the substrate into a container,
(vi) adding extraction solvent and sonicate the container,
(vii) incubating the tube containing the substrate and the extraction solvent for 30 min at room temperature; and
(viii) collecting the supernatant; and with the supernatant, performing mass spectrometry analysis, with or without chromatography, optionally derivatizing the target analyte prior to performing mass spectrometry analysis.
   The method may involve the predetermined volume of the at least one fluid test sample being delivered to a first segment of the substrate, and the method may further comprise delivering a predetermined volume of at least one control sample of the at least one target molecule to a second segment of the substrate; incubating the first segment or a portion thereof with the at least one fluid test sample in a first extraction solvent and incubating the second segment or the portion thereof with the at least one control sample in a second extraction solvent, wherein the first extraction solvent and second extraction solvent are the same or different.

In an alternative embodiment, the method may further comprise delivering a predetermined volume of at least one control sample of the at least one target molecule to a second substrate pre-loaded with a predetermined amount of internal standard molecules such that the at least one control sample contacts the internal standard molecules, the at least one control sample comprising a known concentration of the at least one target molecule; optionally drying the at least one control sample on the second substrate; incubating the second substrate or portion thereof with the at least one control sample in an extraction solvent so as to produce a supernatant for the at least one control sample that comprises the at least one target molecule and the internal standard molecules; detecting the at least one target molecule and the internal standard molecules in the control sample supernatant; generating a calibration curve based on the detection of the at least one target molecule and the internal standard molecules in the control sample supernatant; and quantifying the amount of the at least one target molecule in the test sample based on the amount of the at least one target molecule and the amount of the internal standard molecules detected in the test sample supernatant and correlation with the calibration curve.

In addition, a screening kit is provided for determining the quantity of at least one target molecule in a test sample by using internal standard molecules, such as stable isotope labeled internal standard molecules of a known amount, wherein the stable isotope labeled internal standard molecule is a molecule corresponding to the target molecule labeled with at least one stable isotope. According to this embodiment, the kit may comprise one or more of the following:
(i) at least one substrate,
(ii) at least one of stable isotope labeled internal standard molecules, which can be a concentrated liquid or dried powder form; and/or
(iii) at least one substrate pre-loaded with at least one of stable isotope labeled internal standard molecules of a known amount,
wherein the kit optionally comprises at least one extraction solvent.

According to another embodiment, the kit comprises at least one substrate and at least one of stable isotope labeled internal standard molecules, and further comprises a loading means for loading the at least one substrate with at least one of stable isotope labeled internal standard molecules. Examples of loading means, includes but is not limited to, a syringe, a pipet, a container with a dropper nozzle, and the like.

The screening kit may further comprise instruction sheets including the following steps:
a step of preparation of a reconstitution solution comprising at least one of stable isotope labeled internal standard molecules,
a step of preparation of a working solution comprising at least one reconstituted stable isotope labeled internal standard molecule with the extraction solvent, and/or
steps of using the kit;
(i) load a known amount of working solution comprising at least one of stable isotope labeled internal standard molecules onto a substrate;
(ii) dry the substrate for a predetermined amount of time;
(iii) add a known volume of the test sample onto the substrate preloaded with at least of one stable isotope labeled internal standard molecules,
(iv) dry the substrate sufficiently;
(v) put the substrate into a container (e.g. tube or other suitable container);
(vi) add extraction solvent and sonicate the container;
(vii) incubate the container containing the substrate and the extraction solvent for an appropriate time and temperature (e.g. 30 min at room temperature);
(viii) take the supernatant; and with the supernatant, perform mass spectrometry analysis.

According to another embodiment, provided is a microfluidic device that includes an inlet for application of a fluid test sample; a metering channel arranged in fluid communication with the inlet for receiving at least a portion of the fluid test sample from the inlet, the metering channel having a predetermined volume; and a substrate arranged to receive a metered volume of the fluid test sample from the metering channel, the substrate comprising a predetermined amount of internal standard molecules. In a specific aspect, the substrate is comprised of a liquid-absorbing material. In certain examples, the substrate is directed to a disc made of cotton, paper, fiber cloth, polymer resin or combinations thereof. In other examples, the internal standard molecules optionally comprise a label, the label optionally being a stable isotope. The stable isotope may include but is not limited to 2H, 13C, 15N, 18O, 34S, or any combinations thereof. In a specific example when a stable and labeled isotope is implemented as an internal standard molecule it may be provided in an amount range of 0.1-5 mmol.

In specific embodiments, the stable isotope labeled internal standard molecule is selected from stable isotope labeled amino acids, free carnitine, acylcarnitines, and any combinations thereof, such as 2H4-alanine, 13C6-,15N4-arginine, 13C4-,15N2-asparagine, 2H3-aspartic acid, 13C3-cysteine, 2H3-glutamic acid, 13C5-,15N2-glutamine, 13C-,15N-glycine, 13C6-, 15N3-histidine, 13C6-, 15N-isoleucine, 2H3-leucine, 13C6-,15N2-lysine, 2H3-methionine, 13C6-phenylalanine, 13C5-, 15N-proline, 13C3-serine, 13C4-threonine, 13C11-,15N2-tryptophan, 13C6-tyrosine, 2H8-valine, 2H2-citrulline, 2H6-ornithine, 2H9-carnitine, 2H3-acetylcarnitine, 2H3-propionylcarnitine, 2H3-butyrylcarnitine, 2H9-isovalerylcarnitine, 2H3-glutarylcarnitine, 2H3-hexanoylcarnitine, 2H3-octanoylcarnitine, 2H3-decanoylcarnitine, 2H3-lauroylcarnitine, 2H9-myristoylcarnitine, 2H3-palmitoylcarnitine, and/or 2H3-stearoylcarnitine, and/or any combinations thereof. In a specific example, the stable isotope labeled internal standard molecule is 13C6-phenylalanine.

In both method and microfluidic device embodiments, the substrate may be made from any material that can absorb liquid, including cotton, paper, fiber cloth, polymer resin, and the like; and the extraction solvent comprises a C1-3 linear or branched chain monoalcohol, in particular methanol, acetonitrile, acetone, chloroform, to methy-t-butylether, and the like.

Further, in specific embodiments, the target molecule for detection and analysis according to the teachings and embodiments herein may include, but is not limited to, free carnitine, acylcarnitines and any combinations thereof in a test sample, such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, valine, citrulline, ornithine, free carnitines, acetyl carnitine, propionylcarnitine, butyrylcarnitine, isovalerylcarnitine, glutarylcarnitine, hexanoylcarnitine, octanoylcarnitine, decanoylcarnitine, lauroylcarnitine, myristoylcarnitine, palmitoylcarnitine, and/or stearoylcarnitine, and/or any combinations thereof, and in particular the target molecule is phenylalanine.

Further, the stable isotope labeled internal standard molecule may include, but is not limited to, stable isotope labeled amino acids, free carnitine, acylcarnitines, and any combinations thereof, such as ²H4-alanine, ¹³C6-,¹⁵N4-arginine, ¹³C4-,¹⁵N2-asparagine, ²H3-aspartic acid, ¹³C3-cysteine, ²H3-glutamic acid, ¹³C5-,¹⁵N2-glutamine, ¹³C-,¹⁵N-glycine, ¹³C6-,¹⁵N3-histidine, ¹³C6-,¹⁵N-isoleucine, ²H3-leucine, ¹³C6-,¹⁵N2-lysine, ²H3-methionine, ¹³C6-phenylalanine, ¹³C5-,¹⁵N-proline, ¹³C3-serine, ¹³C4-threonine, ¹³C11-,¹⁵N2-tryptophan, ¹³C6-tyrosine, ²H8-valine, ²H2-citrulline, ²H6-ornithine, ²H9-carnitine, ²H3-acetylcarnitine, ²H3-propionylcarnitine, ²H3-butyrylcarnitine, ²H9-isovalerylcarnitine, ²H3-glutarylcarnitine, ²H3-hexanoylcarnitine, ²H3-octanoylcarnitine, ²H3-decanoylcarnitine, ²H3-lauroylcarnitine, ²H9-myristoylcarnitine, ²H3-palmitoylcarnitine, and/or ²H3-stearoylcarnitine, and/or any combinations thereof, and in particular the stable isotope labeled internal standard molecule is 13C6-phenylalanine.

These and other embodiments are further described below.

### BRIEF DESCRIPTION OF THE DRAWINGS

The detailed description of preferred embodiments of the invention will be better understood when read in conjunction with the appended drawings. For the purpose of illustrating the invention, the drawings show certain, but not all, preferred embodiments. It should be understood that embodiments of the invention are not limited to the precise arrangements and instrumentalities of those shown in the drawings.

FIG. 1 shows certain types of extraction methods. Type A: Aqueous reconstitution and dilution, Type B: Organic solvent extraction. Extraction of small molecules from a Dried Blood Spot (DBS) follows type B (bottom) process where the DBS is punched out, transferred to a vial and organic solvent is added. The internal standard is typically added immediately before the organic solvent is added.

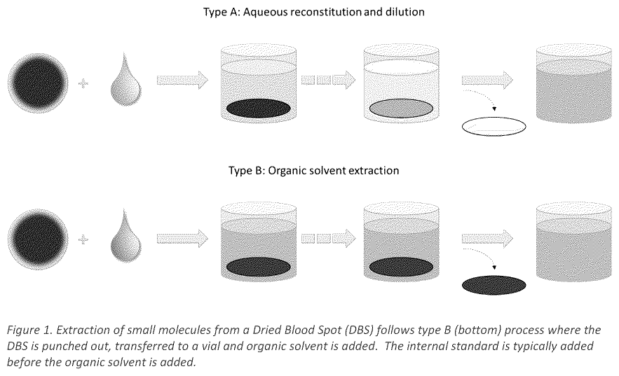

FIG. 2 shows representation of recovery of a small molecule extracted from a DBS card. If there is no loss during extraction, 100% of the small molecule of interest is recovered from the spot (top workflow). However, there is always loss of analyte in the process which results in a lower recovery (middle workflow). Adding an internal standard prior to extraction enables an improved method to achieve near 100% recovery (bottom workflow). The internal standard is represented as A*.

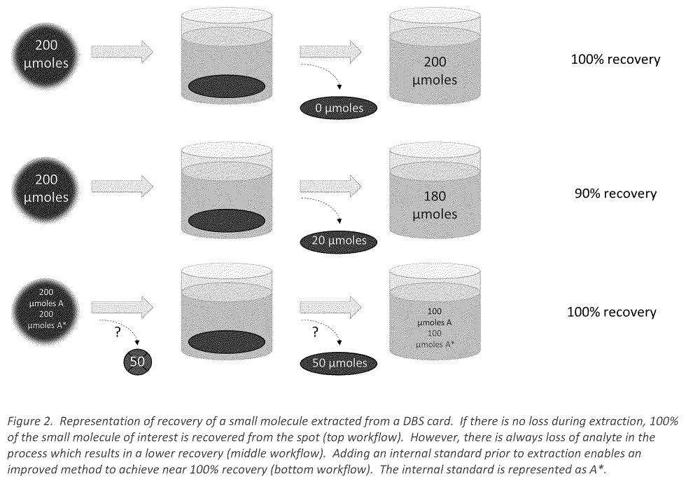

FIG. 3 shows the Stable Isotope Blood Spot (SIBS) process for the quantitation of amino acids. A. The qDBS card is modified to include internal standards (IS). Blood at a specific volume can then be added which will mix with the internal standards to enable simpler and improved extraction and quantitation. B. A representative amino acid profile is shown in the bottom figure.

FIG. 4 shows spectra for phenylalanine and phenylalanine ¹³C6 at different volumes added to the card showing the change in response.

FIG. 5 shows a diagram that illustrates and explains an embodiment of volumetric microsampling.

FIG. 6 shows a diagram of different options for utilization of a control sample. FIG. 6A a relates to a single substrate on which a fluid test sample is delivered to a first segment of the substrate and a control sample is delivered to a second segment of the substrate. FIGS. 6B and 6C shows a second option where a fluid test sample is delivered to a first substrate (FIG. 6B) and a control sample is delivered to a second substrate (FIG. 6C). The substrate is cut where the samples are delivered and then the samples are processed to produce a supernatant for analysis.

Table 1 shows the observed error when the internal standard is added after blood collection compared with when the internal standard is added prior to blood collection (Before).

Table 2 provides examples of volumetric metering or microsampling solutions.

### DETAILED DESCRIPTION

Disclosed herein is process that involves preloading a substrate, the substrate typically used for collecting and quantifying components of a biological sample (e.g. quantitative dried blood spot (qDBS) card), with an amount of a desired internal standard for quantification. In one specific example, the substrate pertains to a cotton disk onto which a precise volume of sample is applied, wherein the substrate already contains known amounts of desired internal standards for quantitation. The preloading of the internal standard onto the substrate facilitates the immediate mixing of the internal standard with the blood sample prior to extraction and enables a near 100% extraction recovery. Providing a substrate preloaded with an internal standard as described herein also reduces lab errors by eliminating the need to prepare internal standards and provides for population screening method and device with improved lab-to-lab reproducibility.

Mass spectrometry is the gold-standard for quantitation of many biochemicals due to its sensitivity, specificity and selectivity. Inherent in quantitative mass spectrometry is the use of stable isotope internal standards. Most internal standards used are stable isotope versions of the targeted compound, e.g. tryptophan with 3 deuterium atoms as a reference for tryptophan. The use of stable isotope internal standards helps to reduce error associated with extraction and ionization efficiencies in different individuals and different biological matrices. In a typical experiment, a volumetric aliquot of biofluid (e.g. plasma) is transferred to a tube and the internal standard is added at a precise volume and concentration. The precise volumes are important for accurate quantitation across a set of samples. Once mixed, the sample is extracted following a desired process that optimizes the recovery of the target analyte(s). Since the internal standard was added at the beginning of the process, any lab errors that occur after this step are easily accounted for since the internal reference standard is present.

To this end, a method as well as a microfluidic device using the pre-loaded internal standards that are to be used as a more reliable reference is provided in this disclosure for the quantitation of target molecules (e.g. amino acids, metabolites, lipids, peptides, proteins, heavy metals, toxins, drugs, or type of analytes) in bio-samples obtained. The method, microfluidic device and kit embodiments may be used for screening for or diagnosing a variety medical condition. And in particular for metabolic disorder screening such as of newborn screening.

In addition, embodiments described herein can also be applied to new fields of use including pharmaceutical drug quantitation, illicit drug quantitation and home health testing. Further, it could also be utilized in clinical trials conducted by pharmaceutical companies which would save them on blood shipping costs and help to standardize the blood collection process across multiple sites. While implementation of the methods taught herein was successfully demonstrated using small molecules, they can readily be adapted for quantitation for peptides and potentially protein analysis.

As is discussed in more detail in the Examples section below, in order to demonstrate the feasibility of the new approach of using-pre-loaded stable isotope labeled internal standards as reference for the quantitation of target molecules, the quantitation of amino acids was shown using stable-isotope labeled analogues. In particular, phenylalanine 13C6 was used as a model compound. The observed error was compared when the internal standard was added after blood collection versus when the internal standard was added before blood collection.

For these examples, a known volume of sample such as blood was collected on a substrate such as cotton paper in a qDBS card, which was pre-loaded with known amounts of internal standards, and dried to obtain a dried blood spot (DBS). For extraction and quantitation of amino acids from DBS, the cotton disk of DBS sample was punched out of the card and extracted with a solvent such as methanol.

In a specific embodiment, disclosed is a method that comprises the steps of
(ix) loading a known amount of solution comprising at least one of stable isotope labeled internal standard molecules onto a substrate,
(x) drying the substrate for a predetermined time, e.g. 2-48 hrs,
(xi) adding a known volume of the fluid test sample onto the substrate preloaded with at least one of stable isotope labeled internal standard molecules;
(xii) drying the substrate sufficiently,
(xiii) putting the substrate into a container,
(xiv) adding extraction solvent and sonicate the container,
(xv) incubating the tube containing the substrate and the extraction solvent for 30 min at room temperature; and
(xvi) collecting the supernatant; and with the supernatant, performing mass spectrometry analysis, with or without chromatography, optionally derivatizing the target analyte prior to performing mass spectrometry analysis.
   The method may involve the predetermined volume of the at least one fluid test sample being delivered to a first segment of the substrate, and the method may further comprise delivering a predetermined volume of at least one control sample of the at least one target molecule to a second segment of the substrate; incubating the first segment or the portion thereof with the at least one fluid test sample in a first extraction solvent and incubating the second segment or the portion thereof with the at least one control sample in a second extraction solvent, wherein the first extraction solvent and second extraction solvent are the same or different. In a more specific embodiment, the substrate includes two or segments upon which predetermined volumes of two or more control samples are delivered. The two or more control samples may include a first volume at a first predetermined concentration of at least one target molecule and a second volume of a second predetermined concentration of at least one target molecule. The first and second volumes of the first and second predetermined concentrations of the at least one target molecule may be the same or different.

In an alternative embodiment, the method may further comprise delivering a predetermined volume of at least one control sample of the at least one target molecule to a second substrate pre-loaded with a predetermined amount of internal standard molecules such that the at least one control sample contacts the internal standard molecules, the at least one control sample comprising a known concentration of the at least one target molecule; optionally drying the at least one control sample on the second substrate; incubating the second substrate or portion thereof with the at least one control sample in an extraction solvent so as to produce a supernatant for the at least one control sample that comprises the at least one target molecule and the internal standard molecules; detecting the at least one target molecule and the internal standard molecules in the control sample supernatant; generating a calibration curve based on the detection of the at least one target molecule and the internal standard molecules in the control sample supernatant; and quantifying the amount of the at least one target molecule in the test sample based on the amount of the at least one target molecule and the amount of the internal standard molecules detected in the test sample supernatant and correlation with the calibration curve. In a more specific embodiment, the method may involve delivering at least one control sample to a third substrate, wherein the concentration of the at least one target molecule in the at least one control delivered to the third substrate is different than the concentration of the at least one target molecule in the at least one control sample delivered to the second substrate. As described above for the second substrate, the at least one control sample delivered to the third substrate can be incubated in an extraction solvent so as to produce a supernatant for the at least one control sample that comprises the at least one target molecule and the internal standard molecules; and the at least one target molecule and the internal standard molecules on the third substrate are detected in the control sample supernatant from the third substrate. Generating a calibration curve may be based on the detection of the at least one target molecule and the internal standard molecules in the control sample supernatant from the second substrate and the at least one target molecule and the internal stand molecules in the control sample supernatant from the third substrate.

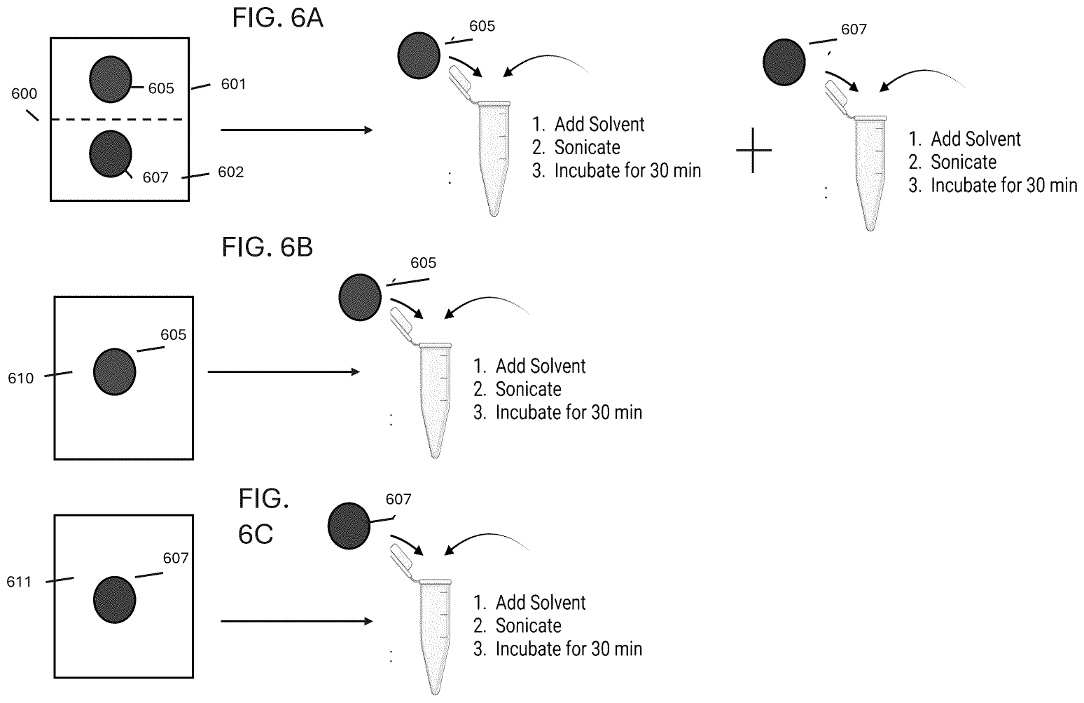

FIG. 6 provides a diagram of the methods that involve implementation of a control sample. As shown in FIG. 6A, a substrate 600 has two different segments 601 and 602. A test sample 605 (e.g. blood) is delivered to segment 601 and a control sample 607 is delivered to segment 609. After delivery, the test sample 605 on the first segment 601 of the substrate 600 and the control sample 607 on the second segment 602 of the substrate is extracted as described herein. The full segment or a portion thereof containing the sample may be used for extraction. FIG. 6B shows a substrate 610 onto which a test sample 605 is delivered and FIG. 6C shows a substrate 611 onto which a control sample is delivered. The test sample 605 on substrate 610 and the control sample on substrate 611 are extracted as described herein. 610 It is noted that when two or more substrates are implemented, or a multi-segmented card is implemented, the substrates can be housed in the same microfluidic device (e.g. qDBS card) that is equipped with separate channels that deliver the respective samples to the different cards or different segments.

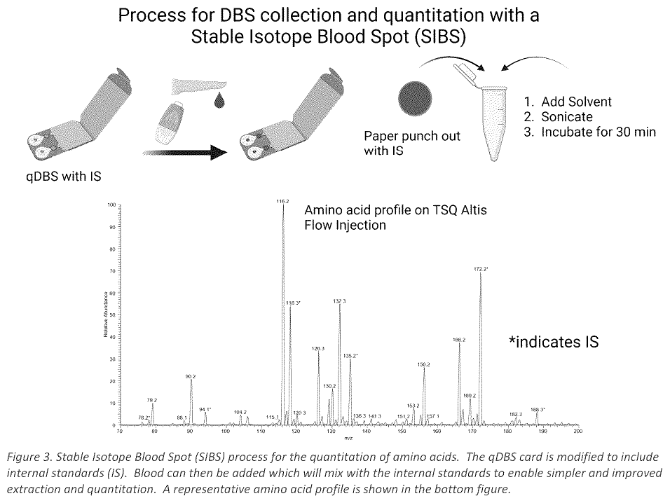

In one embodiment, the process of quantitation using stable isotope blood spot (SIBS) on the substrate is presented, and the resulting spectra of quantitative analysis of the SIBS showed the presence of stable isotope labeled amino acids along with their endogenous counterparts (FIG. 3).

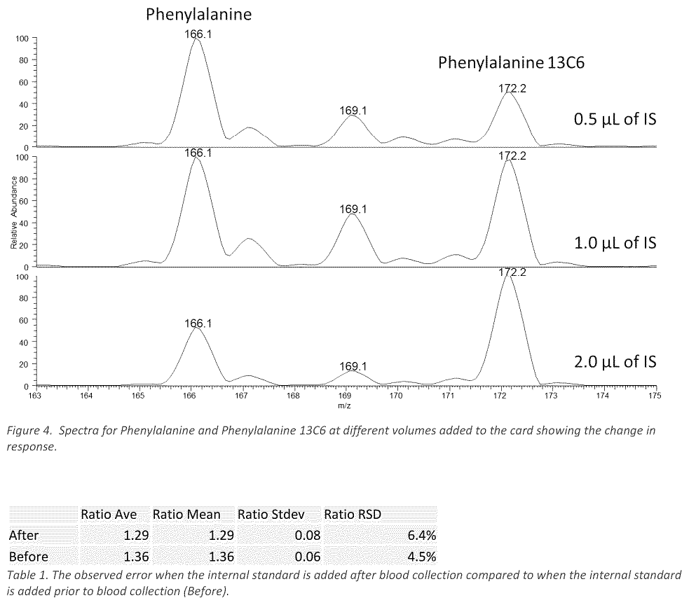

In another embodiment, phenylalanine ¹³C6 was used to quantitate an amino acid, phenylalanine. Phenylalanine is the amino acid of which accumulation is related to the onset of a rare inherited metabolic disorder, phenylketonuria (PKU). PKU is screened by newborn screening tests, and can be reliably diagnosed by detecting the level of phenylalanine in blood. Here, different volumes of Phenylalanine ¹³C6 was pre-loaded as an internal standard of phenylalanine prior to the addition of blood onto the cotton disk. FIG. 4 shows that when the volume of internal standard of ¹³C6 phenylalanine and hence the quantity of it were changed, the ratio of relative abundance of phenylalanine vs. phenylalanine ¹³C6 changed.

In another embodiment, the observed errors when the internal standard was added after blood collection and when the internal standard was added before blood collection were shown in Table 1. As a result, the standard deviation and relative standard deviation (RSD) of the ratio were smaller when the internal standard was added before blood collection than those when the internal standard was added after blood collection.

The data provided herein demonstrates that the discovered approach of pre-loading of stable isotope labeled internal standards is well suited for the quantitation of a broad swath of analytes.

### Definitions

The preferred materials and methods are described herein; any methods and materials similar or equivalent to those described herein can be used in the practice of or testing of the invention. Unless defined otherwise, all technical and scientific terms herein have the same meaning as commonly understood by one of ordinary skill in the art to which the invention pertains. In describing and claiming the present invention, the following terminology will be used. It is also to be understood that the terminology used herein is for the purpose of describing particular embodiments only, and it is not intended to be limiting.

The articles “a,” “an,” “the” and the like are used herein to refer to one or to more than one (i.e., to at least one) of the grammatical object of the article, unless specifically noted otherwise. By way of example, “an element” means one element or more than one element. Unless otherwise indicated, “or” encompasses “and.” To illustrate, “A, B, or C” means A alone, B alone, C alone, the combination of A and B, the combination of A and C, the combination of B and C, and the combination of A, B, and C, unless otherwise illustrated.

It is to be noted that the terms “first,” “second,” and the like as used herein do not denote any order, quantity, or importance, but rather are used to distinguish one element from another, unless otherwise specifically stated herein. The terms “a” and “an” do not denote a limitation of quantity, but rather denote the presence of at least one of the referenced item. The modifier “about” used in connection with a quantity is inclusive of the stated value and has the meaning dictated by the context. The term “about” generally refers to a range of numbers that one of skill in the art would consider equivalent to the recited value (i.e., having the same function or result). In a specific embodiment, the term “about” includes a stated numerical value as well as a value that is +/−30% of the stated numerical value. For example, about 40 degrees includes 40 degrees as well as angles of 36 degrees and 44 degrees, and all values in between. In a further specific embodiment, the term “about” includes a stated numerical value as well as a value that is +/−25%. In many instances, the term “about” may include numbers that are rounded to the nearest significant figure. Furthermore, to the extent that the terms “including,” “includes,” “having,” “has,” “with,” or variants thereof are used in either the detailed description and/or the claims, such terms are intended to be inclusive in a manner similar to the term “comprising.”

The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

As used herein, “amino acid” refers to an organic compound that contains both amino- and carboxylic functional groups. There are hundreds of amino acids in nature, and they can be classified according to the structure based on the locations of the core functional groups into alpha- (α-), beta- (β-), gamma- (γ-) or delta- (δ-) amino acids. Alpha- (α-) amino acid is an amino acid having the amino- and carboxylic functional groups attached to the same carbon atom (α-carbon) and occurs naturally in peptides and proteins, including proline and hydroxyproline, which are secondary amines. Due to this structure, all α-amino acids have chirality with the exception of achiral glycine. In this disclosure, the amino acid refers to all L/D isomers and S/R enantiomers. Beta- (β-), gamma- (γ-) or delta- (δ-) amino acids are non-proteinogenic amino acids in which the amino group is located on the carbon atom at the position β, γ, or δ to the carboxyl group.

Proteogenic alpha-amino acids can be classified according to the structure and ionization of their side chains into nonpolar aliphatic amino acids such as glycine (Gly, G), alanine (Ala, A), valine (Val, V), leucine (Leu, L), isoleucine (Ile, I) and proline (Pro, P); polar neutral amino acids such as serine (Ser, S) and threonine (Thr, T); sulfur-containing amino acids such as cysteine (Cys, C) and methionine (Met, M); aromatic amino acids such as phenylalanine (Phe, F), tyrosine (Tyr, Y) and tryptophan (Trp, W); amide amino acids such as asparagine (Asn, N) and glutamine (Gln, Q); anionic amino acids such as aspartate (“aspartic acid”, Asp, D) and glutamate (“glutamic acid”, Glu, E); and cationic amino acids such as histidine (His, H), lysine (Lys, K) and arginine (Arg, R). These proteogenic 20 amino acids are standard/canonical amino acids encoded by the universal genetic code, and they are joined by peptide bond which is an amide type covalent bond formed by joining C1 of the carboxyl group of one amino acid to the amino group of another, linking two consecutive alpha-amino acids to form a linear and unbranched polymer chain called peptide. Other amino acids are non-proteogenic and called nonstandard/non-canonical except two amino acids, i.e., selenocysteine and pyrrolysine. Although these two nonstandard/non-canonical amino acids are incorporated into peptides or proteins very rarely, they can be incorporated translationally by exploiting information not encoded in the universal genetic code. There are many known non-proteinogenic and nonstandard/non-canonical amino acids (e.g., carnitine, gamma-aminobutyric acid, levothyroxine, hydroxyproline, selenomethionine, etc.). Some of non-proteinogenic amino acids are found in proteins, and they are formed by post-translational modification, which are often essential for the function or regulation of a protein (e.g., carboxyglutamic acid/carboxyglutamate, hydroxyproline, hypusine, etc.)

Other non-proteinogenic amino acids are not found in proteins (e.g., gamma-aminobutyric acid neurotransmitter), and some of them often occur as intermediates in the metabolic pathways for standard amino acids (e.g., ornithine and citrulline in the urea cycle), part of amino acid catabolism. The amino acids in this disclosure comprise all of the amino acids described above.

As used herein, “carnitine” refers to a quaternary ammonium compound involved in energy metabolism in most mammals. Generally, carnitine is both a nutrient and synthesized by the body as needed. The key role of carnitine is to serve as a carrier for the transportation of long-chain fatty acids into mitochondria to generate energy through ß-oxidation. Considering its metabolic role, carnitine is concentrated in tissues like skeletal and cardiac muscles that metabolize fatty acids as an energy source.

Free carnitine and fatty acylcarnitine can diffuse freely from the cytosol across the porous outer mitochondrial membrane to the intermembrane space, but they need carnitine-acylcarnitine translocase (CACT) to cross the nonporous inner mitochondrial membrane to reach the mitochondrial matrix where β-oxidation takes place. CACT is a shuttle system for passive transport of carnitine and fatty acylcarnitine, functioning by transporting one molecule of free carnitine from the matrix to the intermembrane space while transporting one molecule of fatty acylcarnitine from the intermembrane space into the matrix. The deficiency of this protein prevents the body from using certain fats for energy, particularly during periods without food (fasting).

The acylcarnitine profile is a diagnostic test for inherited metabolic disorders of fatty acid as well as branched-chain amino acid catabolism patients with this type of metabolic disorder accumulate disease-specific acylcarnitines that correlate with the acyl coenzyme A compounds in the affected mitochondrial metabolic pathways. Newborns with high levels of these substances might have CACT deficiency, which might be due to mutations in the SLC25A20 gene. Signs and symptoms of this disorder usually begin soon after birth and may include breathing problems, seizures, and an irregular heartbeat (arrhythmia). Affected individuals typically have low blood sugar (hypoglycemia) and a low level of ketones, which are produced during the breakdown of fats and used for energy. Together these signs are called hypoketotic hypoglycemia. People with CACT deficiency also usually have excess ammonia in blood (hyperammonemia), an enlarged liver (hepatomegaly), and a weakened heart muscle (cardiomyopathy). Many infants with CACT deficiency do not survive the newborn period. Some affected individuals have a less severe form of the condition and do not develop signs and symptoms until early childhood. These individuals are at risk for liver failure, nervous system damage, coma, and sudden death.

Some babies do not have enough carnitine due to carnitine uptake defect (CUD), also known as primary carnitine deficiency. CUD is an autosomal recessive disorder caused by mutations of the SLC22A5 gene, which encodes the high-affinity carnitine transporter OCTN2 in the plasma membrane. CUD results in non-absorbed carnitine to be wasted in urine and consequently systemic and intracellular carnitine deficiencies, which eventually leads to defects in beta-oxidation of fatty acids. CUD is a potentially lethal disease, and patients with CUD usually have early-onset cardiomyopathy (including poor contractility, thickened ventricular walls or increased T waves on EKG), muscle weakness, recurrent hypoketotic hypoglycemic coma or Reye-like syndrome. Laboratory evaluations reveal extremely low carnitine concentrations in blood and tissue (<5% of normal), and with early treatment most symptoms are reversible.

As used herein, a “metabolic disorder” refers to a disorder that negatively alters the body's metabolism i.e., the ongoing biochemical reactions for the maintenance of the balance of two processes: catabolism that is the set of chemical reactions of breaking down larger molecules into smaller ones to generate energy, such as breaking down carbohydrate molecules into glucose, and anabolism (i.e., biosynthesis) that is the set of chemical reactions of constructing molecules from smaller units by consuming energy. A metabolic disorder occurs when an abnormal chemical reaction in the body alters or disrupts these processes. As a result of abnormal chemical reactions, there might be too much of some substances or too little of others, and this impairs protein-, fat- and carbohydrate metabolism and/or affects various organelle functions leading to complicated medical conditions.

There are different groups of metabolic disorders, and among them inherited metabolic disorders are genetic conditions, most of which are autosomal recessive. Most inherited metabolic disorders are caused by a single mutation of a gene, which consequently encodes a missing/defective enzyme, i.e., the enzyme is either not produced at all by the body or is produced in a malfunctional form, which can cause toxic compound accumulation or lack of an essential product (e.g., urea cycle defect, amino acid disorders such as phenylketonuria (PKU), maple syrup urine disease (MSUD)), abnormal energy generation or consumption (e.g., mitochondrial disorders, glycogen metabolism disorders, glycogen storage diseases, galactosemia, fatty acid oxidation disorders), and/or defective organelle functions (e.g., lysosomes, peroxisomes, Golgi and endoplasmic reticulum). There are hundreds of identified genetic/inherited metabolic disorders, and new ones are continuously being discovered.

Children and adolescents with inherited metabolic disorders have a wide spectrum of clinical presentations from appearing physically normal to having distinctive dysmorphic physical features. While majority of them appear physically normal at birth, many can present with significant non-specific signs and symptoms common to other serious medical conditions. While each inherited metabolic disorder is quite rare in the general population, considered all together, their cumulative incidence is relatively high, around 1 in 1,000 to 2,500 newborns.

As used herein, “newborn screening (NBS)” refers to a public health program of screening in infants shortly after birth for conditions that are treatable, but not clinically evident in the newborn period. The goal is to identify infants at risk for these conditions early enough to prevent or ameliorate the clinical symptoms. Newborn screening is performed with a few drops of blood obtained by pricking the baby's heel. Target disorders of newborn screening include: metabolic disorders such as amino acid disorders, urea cycle disorders, fatty acid oxidation disorders, organic acidemias and lysosomal storage disorders, endocrinopathies, hemoglobinopathies, cystic fibrosis, congenital heart defects, severe combined immunodeficiency, hearing loss, and other conditions.

As used herein, an “organic acid” refers to an organic compound with acidic properties. The most common organic acids are the carboxylic acids having carboxyl group —COOH, which is a relatively weak acid. The acidity is determined by the relative stability of the conjugate base of the acid. For example, sulfonic acids, having the group —SO₂OH, are relatively stronger acids. There are many other groups of weak acidity, having the thiol group —SH, the enol group, and the phenol group. In biological systems, organic compounds containing these groups are generally referred to as organic acids. A few common examples are lactic acid, acetic acid, formic acid, citric acid, oxalic acid, uric acid, malic acid, tartaric acid, etc.

As used herein, “phenylketonuria (PKU)” refers to an inherited, autosomal recessive metabolic disorder, which is conventionally caused by lack of the enzyme, phenylalanine hydroxylase (PAH). PAH is an enzyme that catalyzes the hydroxylation of the aromatic ring of phenylalanine to generate tyrosine, which is converted to a variety of important compounds, including thyroxine, norepinephrine, epinephrine and melanin, or is converted to p-hydroxyphenylpyruvate to be eventually degraded to acetoacetate and fumarate.

The hydroxylation reaction of phenylalanine occurs principally in the liver but also in the kidney, and it requires dihydropteridine reductase and tetrahydrobiopterin in addition to phenylalanine hydroxylase. The hydroxylation of phenylalanine occurs through coupled oxidation/reduction reactions consisting of oxidation of phenylalanine to tyrosine and of tetrahydrobiopterin to a quinonoid dihydrobiopterin with molecular oxygen as the electron acceptor: the tetrahydrobiopterin is regenerated by the reduction of the quinonoid dihydrobiopterin in the presence of NAD(P)H by dihydropteridine reductase:

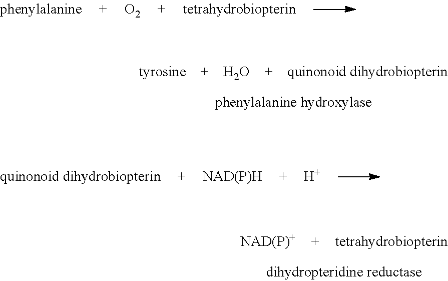

When phenylalanine hydroxylase is deficient or absent, phenylalanine is not converted to tyrosine. Instead, it accumulates in the circulation and is converted by glutamine-phenylpyruvate transaminase to phenylpyruvate, a phenyl ketone that is eventually excreted in the urine. In normal condition, levels of phenylpyruvate are very low in blood or urine; however in PKU, levels of phenylpyruvic acid as well as phenyllactate and phenylacetate are very high in the urine, sweat, breath causing a slightly musty odor.

Other than the deficiency of PAH, deficiency of dihydropteridine reductase and tetrahydrobiopterin can also cause a substantial decrease in the rate of phenylalanine hydroxylation. Tetrahydrobiopterin synthesis starts with GTP and requires reactions mediated by GTP-cyclohydrolase, 6-Pyruvoyltetrahydrobiopterin synthase and sepiapterin reductase. Deficiency of these three enzymes also causes hyperphenylalanemia (high concentration of phenylalanine in blood).

Phenylalanine is a large, neutral amino acid which moves across the blood-brain barrier (BBB) via the large neutral amino acid transporter (LNAAT). Excessive phenylalanine in blood saturates the transporter, and thus significantly decreases the levels of other large neutral amino acids in the brain. But since these amino acids are required for protein and neurotransmitter synthesis, phenylalanine accumulation disrupts brain development, leading to microcephaly and mental retardation. If untreated, the symptoms such as unusual irritability, hyperactivity, epileptic seizures and skin lesions become evident by the first year, and in addition to these symptoms, EEG abnormalities and severe learning disabilities are major clinical problems later in life.

As used herein, “qDBS card” refers to a quantitative dried blood spot (qDBS) card such as Capitainer® qDBS, which is a card for blood sample collection.

### Volumetric Metering

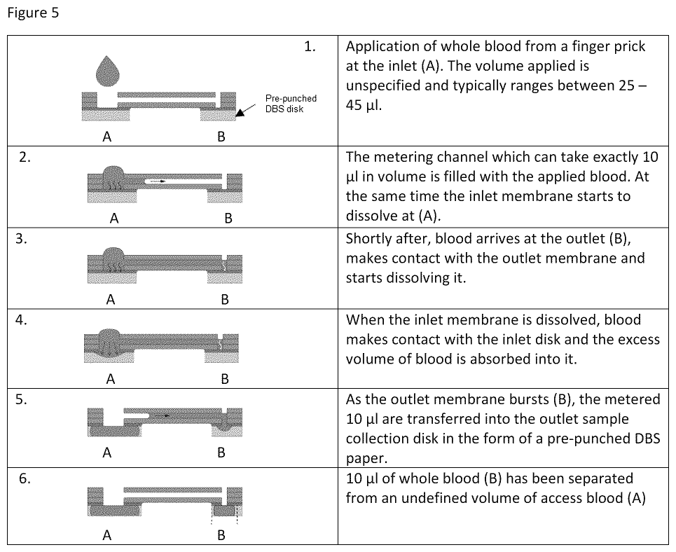

The accuracy of detecting metabolites using methods and microfluidic devices described herein is directly related to the ability to apply precise volumes of sample to a substrate preloaded with an internal standard. In one embodiment, the CAPITAINER® B card microsampling system is implemented. Such a microfluidic device is described in WO2015044454A2, the contents of which is incorporated herein by reference. The CAPITAINER® B card uses a combination of paper and polymer microfluidics to meter a 10 μl fixed volume from an undefined volume of finger prick blood. The operation of this system is illustrated and explained in FIG. 5. After applying a drop of blood to the inlet, a metering channel in the device is automatically filled. A valve composed of a thin dissolvable membrane then opens up and removes excessive blood at the inlet. Subsequently, a second membrane opens up at the outlet, allowing for transfer of the metered blood volume onto the sample collection disk. The sample is allowed to dry forming a high quality DBS sample suitable for quantitative analysis.

Those skilled in the art will appreciate that embodiments described herein of preloading internal standards can be applied to other volumetric microsampling systems known in the field. While the vast majority of the microsampling market consists of solutions for non-quantitative (traditional DBS) and semi-quantitative sampling systems, there are commercially available solutions for volumetric microsampling. Table 2 lists such commercially available solutions for sampling a fixed volume of whole blood.

TABLE 2

| | | | |
|---|---|---|---|
| Company/ | Volume | Volume | |
| Product name | definition | performance | Metering technology |

FINGER PRICK COLLECTION

| | | | |
|---|---|---|---|
| Capitainer AB | yes | Better than 0.5 μl | Metering channel connected to pre-punched paper |
| Capitainer ® B | | as one Standard | disks coated with thin water dissolvable |
| | | Deviation (SD), | membranes (Polyvinyl alcohole) as described |
| | | (CV <5%) | earlier. |
| DBS Systems | yes | Volume per | Metering channel absorbs a fixed volume. The |
| HemaXis DB | | blood spot: | volume is transferred to a DBS paper through a |
| | | 10.0 μL ± 0.5 μL | folding mechanism that allows the opening of the |
| | | (95% confidence | metering channel to make contact with a paper. |
| | | interval) | The capillary force of the paper extracts the |
| | | | metered volume from the channel. The metered |
| | | | dried blood volumes are punched out from the |
| | | | paper at the laboratory and further processed. |
| Neoteryx | yes | <4% CV | VAMS (Volumetric Absorbtive microsampling). |
| Mitra | | | Q-tip-like concept. Polymer tip with a defined |
| | | | absorptive volume capacity is immersed into the |
| | | | blood drop whereby the blood is absorbed into it |
| | | | until its saturated. |
| Trajan | yes | <2.75% CV | Glass capillaries meters a volume which is |
| Hemapen | | | transferred to a pre-punched DBS paper disks. |
| Weavr health | yes | None reported | Metering via glass capillaries that are transferred to |
| Velvet | | | paper. Lateral flow separation into plasma. Samples |
| | | | are punched out from the paper at the laboratory |

UPPER ARM DEVICES

| | | | |
|---|---|---|---|
| Seven Sense | semi | Liquid sample | Upper arm device. Small plastic container is |
| TAP | | which is | attached to the arm whereby small needles |
| | | pipetted at lab | penetrate the skin. The under-pressure in the device |
| | | | cause blood to be sucked into its cavity. The |
| | | | sampled volume is shipped to the laboratory where |
| | | | its pipetted into smaller volumes for each dedicated |
| | | | assay. |
| Tasso | yes | <5% | Upper arm device like above but optional adaptor |
| Tasso-M20 | | | with volumetric adsorption onto DBS paper “sample |
| | | | pod”. |
| Drawbridge | Unknown | | Same as above. Extracts blood onto paper strip. |
| Health | | | (Volume performance not known.) |
| OneDraw | | | |
| Loop Medical | Unknown | | Like above but contains blood in liquid format. |
| Loop medical | | | |
| One | | | |

### Examples

Embodiments of the invention are further described in detail by reference to the following experimental examples. These examples are provided for purposes of illustration only, and they are not intended to be limiting unless otherwise specified. Thus, the invention should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present invention and practice the claimed methods. Therefore, the following working examples specifically point out the preferred embodiments of the present invention, and are not to be construed as limiting in any way the remainder of the disclosure.

### Materials and Methods

A standard qDBS card was provided by Capitainer. Due to blood viscosity, it is difficult to obtain same volume of blood for a spot. To overcome this problem, a microfluidic quantitative DBS (qDBS) card was used to deliver an exact sample volume to a pre-cut DBS disk. In typical versions, the qDBS card delivers exactly 10 uL of whole blood to a cotton disk. The qDBS card was open to reveal the cotton disks. We added a mixture of stably-labeled amino acids at varying volumes (0.5 uL, 1.0 uL, and 2.0 uL). The card was then dried overnight. Blood was then added to the card from a fingerstick. The card was again allowed to dry which replicates the process by which an individual would use the process. After sufficient drying, the card is punched out, added to a tube, and extracted with organic solvent. After incubating for 30 min, the resulting supernatant was subject to precursor ion scanning for amino acids (looking for a loss of 46) on a triple quadrupole mass spectrometer operating in positive electrospray ionization mode.

### Results

FIG. 3 provides a diagram outlining a process of quantitation using stable isotope blood spot (SIBS) on the substrate and the result of quantitative analysis of amino acids in the SIBS. A mixture of stably-labeled amino acids was loaded onto the cotton disk of qDBS card. After drying the cotton disk overnight, a drop of blood was added to the card from a fingerstick. The cotton disk in the card was dried, and it was punched out and put into a tube for extraction with organic solvent. The resulting spectra showed the presence of stable isotope labeled amino acids along with their endogenous counterparts. The resulting spectra showed the presence of the stably-labeled amino acids along with their endogenous counterparts.

As shown in FIG. 4, phenylalanine ¹³C6 was used to quantitate phenylalanine in the DBS. Here, varying volumes of phenylalanine ¹³C6 (0.5 uL, 1.0 uL, and 2.0 uL) were pre-loaded onto the cotton disk of qDBS card as an internal standard prior to the addition of blood onto the cotton disk. When the volume of internal standard of ¹³C6 phenylalanine and hence the quantity of it were changed, the ratio of relative abundance of phenylalanine vs. phenylalanine ¹³C6 changed.

Moreover, the observed error when the internal standard was added after blood collection was compared with that when the internal standard was added before blood collection. Table 2 shows that the standard deviation and relative standard deviation (RSD) of the ratio were compared between the process in which the internal standard was added before blood collection and the process in which the internal standard was added after blood collection. In this comparison, the standard deviation was 0.08 in after-loading of the internal standard and 0.06 in pre-loading of the internal standard; and the relative standard deviation was 6.4% in after-loading of the internal standard and was improved to 4.5% in pre-loading of the internal standard, proving that pre-loading of internal standards is more reliable method of amino acid quantitation.

In conclusion, these examples showed that this approach of pre-loading of stable isotope labeled internal standards is well suited for the quantitation of small molecules such as amino acids.

### REFERENCES RELATED TO METABOLOMICS

1. Goodacre R. Metabolomics of a superorganism. The Journal of nutrition. 2007; 137:259S-66S. PubMed PMID: 17182837.
2. Kaddurah-Daouk R, Kristal B S, Weinshilboum R M. Metabolomics: A Global Biochemical Approach to Drug Response and Disease. Annual Review of Pharmacology and Toxicology. 2008; 48(1):653-83. doi: 10.1146/annurev.pharmtox.48.113006.094715. PubMed PMID: 18184107.
3. Shevchenko A, Simons K. Lipidomics: coming to grips with lipid diversity. Nature reviews Molecular cell biology. 2010; 11(8):593-8.
4. Nicholson J K, Lindon J C. Systems biology: Metabonomics. Nature. 2008; 455:1054-6. doi: 10.1038/4551054a. PubMed PMID: 18948945.
5. Rai S K, Bril F, Hatch H M, Xu Y, Shelton L, Kalavalapalli S, Click A, Lee D, Beecher C, Kirby A, Kong K, Trevino J, Jha A, Jatav S, Kriti K, Luthra S, Garrett T J, Guingab-Cagmat J, Plant D, Bose P, Cusi K, Hromas R A, Tischler A S, Powers J F, Gupta P, Bibb J, Beuschlein F, Robledo M, Calsina B, Timmers H, Taieb D, Kroiss M, Richter S, Langton K, Eisenhofer G, Bergeron R, Jr., Pacak K, Tevosian S G, Ghayee H K. Targeting pheochromocytoma/paraganglioma with polyamine inhibitors. Metabolism: clinical and experimental. 2020; 110:154297. Epub 2020/06/21. doi: 10.1016/j.metabol.2020.154297. PubMed PMID: 32562798; PMCID: PMC7482423.
6. Taylor N J, Gaynanova I, Eschrich S A, Welsh E A, Garrett T J, Beecher C, Sharma R, Koomen J M, Smalley K S M, Messina J L, Kanetsky P A. Metabolomics of primary cutaneous melanoma and matched adjacent extratumoral microenvironment. PLOS One. 2020; 15(10):e0240849. Epub 2020/10/28. doi: 10.1371/journal.pone.0240849. PubMed PMID: 33108391; PMCID: PMC7591037 following competing interests: Chris Beecher is the Founder and Chief Science Officer of IROA Technologies. Timothy J. Garrett is a member of the Scientific Advisory Board of IROA Technologies. IROA Long Term Reference Standard and Internal Standard were donated by IROA Technologies. Timothy J Garrett's laboratory received no funding from IROA Technologies, and this specific commercial affiliation does not alter the authors' adherence to PLOS ONE policies on sharing data and materials.
7. O'Kell A L, Garrett T J, Wasserfall C, Atkinson M A. Untargeted metabolomic analysis in naturally occurring canine diabetes mellitus identifies similarities to human Type 1 Diabetes. Sci Rep. 2017; 7(1):9467. Epub 2017/08/27. doi: 10.1038/s41598-017-09908-5. PubMed PMID: 28842637; PMCID: PMC5573354.
8. Antharam V C, McEwen D C, Garrett T J, Dossey A T, Li E C, Kozlov A N, Mesbah Z, Wang G P. An Integrated Metabolomic and Microbiome Analysis Identified Specific Gut Microbiota Associated with Fecal Cholesterol and Coprostanol in Clostridium difficile Infection. PloS one. 2016; 11:e0148824. doi: 10.1371/journal.pone.0148824. PubMed PMID: 26871580.
9. Xu R, Wang Q. Towards understanding brain-gut-microbiome connections in Alzheimer's disease. BMC Systems Biology. 2016; 10(3):63. doi: 10.1186/s12918-016-0307-y.
10. Chace D H, Kalas T A, Naylor E W. Use of tandem mass spectrometry for multianalyte screening of dried blood specimens from newborns. Clinical chemistry. 2003; 49:1797-817. PubMed PMID: 14578311.
11. Seger C, Salzmann L. After another decade: LC-MS/MS became routine in clinical diagnostics. Clinical Biochemistry. 2020; 82:2-11. doi: https://doi.org/10.1016/j.clinbiochem.2020.03.004.
12. Cantor J R, Sabatini D M. Cancer cell metabolism: one hallmark, many faces. Cancer discovery. 2012; 2(10):881-98. Epub 2012/09/27. doi: 10.1158/2159-8290.cd-12-0345. PubMed PMID: 23009760; PMCID: PMC3491070.
13. DeBerardinis R J, Chandel N S. Fundamentals of cancer metabolism. Science Advances. 2016; 2(5):e1600200. doi: 10.1126/sciadv.1600200.
14. Reznik E, Luna A, Aksoy B A, Liu E M, La K, Ostrovnaya I, Creighton C J, Hakimi A A, Sander C. A Landscape of Metabolic Variation across Tumor Types. Cell systems. 2018; 6(3):301-13.e3. Epub 2018/02/06. doi: 10.1016/j.cels.2017.12.014. PubMed PMID: 29396322; PMCID: PMC5876114.
15. Vander Heiden M G, Cantley L C, Thompson C B. Understanding the Warburg Effect: The Metabolic Requirements of Cell Proliferation. Science. 2009; 324(5930): 1029-33. doi: 10.1126/science.1160809.
16. DeBerardinis R J, Lum J J, Hatzivassiliou G, Thompson C B. The biology of cancer: metabolic reprogramming fuels cell growth and proliferation. Cell metabolism. 2008; 7(1):11-20. Epub 2008/01/08. doi: 10.1016/j.cmet.2007.10.002. PubMed PMID: 18177721.
17. Hsu P P, Sabatini D M. Cancer cell metabolism: Warburg and beyond. Cell. 2008; 134(5):703-7. Epub 2008/09/09. doi: 10.1016/j.cell.2008.08.021. PubMed PMID: 18775299.
18. Hanahan D, Weinberg R A. Hallmarks of cancer: the next generation. Cell. 2011; 144(5):646-74. Epub 2011/03/08. doi: 10.1016/j.cell.2011.02.013. PubMed PMID: 21376230.
19. Luengo A, Gui D Y, Vander Heiden M G. Targeting Metabolism for Cancer Therapy. Cell chemical biology. 2017; 24(9): 1161-80. Epub 2017/09/25. doi: 10.1016/j.chembiol.2017.08.028. PubMed PMID: 28938091; PMCID: PMC5744685.

### REFERENCES RELATED TO INBORN ERRORS OF METABOLISM

Pourfarzam M and Zadhoush F, Newborn Screening for inherited metabolic disorders; news and views, J Res Med Sci. 2013, 18(9): 801-808.
Agana M, Frueh J, Kamboj M, Patel D R and Kanungo S, Common metabolic disorder (inborn errors of metabolism) concerns in primary care practice, Annals of translational Medicine 2018, 6 (24) (December 2018)
Lee, N C, Tang N L S, Chien Y H et al., Diagnoses of newborns and mothers with carnitine uptake defects through newborn screening, Molecular Genetics and Metabolism 2010, 100 (1): 46-50
Longo N, Primary Carnitine Deficiency and Newborn Screening for Disorders of the Carnitine Cycle, Ann Nutr Metab. 2016, 68 Suppl 3:5-9
Dobrowolski S F, Phua Y L, Vockley J et al., Phenylketonuria oxidative stress and energy dysregulation: Emerging pathophysiological elements provide interventional opportunity, Mol Genet Metab. 2022, 136(2):111-117.
Parra A, García M I, Waisbren S E et al., Cognitive functioning in mild hyperphenylalaninemia, Mol Genet Metab Rep. 2015, 5: 72-75.

1. A method for determining the quantity of at least one target molecule in a fluid test sample, the method comprising:
delivering a predetermined volume of the fluid test sample to a substrate pre-loaded with a predetermined amount of internal standard molecules such that the predetermined volume of the fluid test sample contacts the internal standard molecules;
optionally drying the fluid test sample on the substrate;
incubating the substrate or a portion thereof with the fluid test sample in an extraction solvent so as to produce a supernatant that comprises the at least one target molecule and the internal standard molecules;
detecting the at least one target molecule and the internal standard molecules in the supernatant; and
quantifying the amount of the at least one target molecule in the fluid test sample based on the amount the at least one target molecule and the amount of the internal standard molecules detected in the supernatant, wherein the substrate is, optionally, comprised of a liquid-absorbing material.

2. The method of claim 1, wherein the internal standard molecules comprise a label, the label optionally being a stable isotope label.

3. The method of claim 2, wherein the stable isotope label is selected from the group consisting of ²H, ¹³C, ¹⁵N, ¹⁸O, ³⁴S, or any combinations thereof, and, optionally, wherein predetermined amount of the internal standard molecules is in an amount range of 0.1-5 mmol.

4. (canceled)

5. The method of claim 1, further comprising
loading a predetermined amount of solution comprising internal standard molecules onto the substrate, and
drying the substrate to produce a pre-loaded substrate.

6. (canceled)

7. (canceled)

8. The method of claim 1, wherein the fluid test sample is delivered to the substrate using a microfluidic device comprising a metering channel with a predetermined volume, wherein the substrate is arranged to receiving a metered volume of the fluid test sample from the metering channel, the method further comprising removing the substrate from the microfluidic device.

9. The method of claim 1, wherein the predetermined volume of the at least one fluid test sample is delivered to a first segment of the substrate, and wherein the method further comprises delivering a predetermined volume of at least one control sample of the at least one target molecule to a second segment of the substrate; incubating the first segment or a portion thereof with the at least one fluid test sample in a first extraction solvent and incubating the second segment or a portion thereof with the at least one control sample in a second extraction solvent, wherein the first extraction solvent and second extraction solvent are the same or different.

10. The method of claim 1, further comprising:
delivering a predetermined volume of at least one control sample of the at least one target molecule to a second substrate pre-loaded with a predetermined amount of internal standard molecules such that the at least one control sample contacts the internal standard molecules, the at least one control sample comprising a known concentration of the at least one target molecule;
optionally drying the at least one control sample on the second substrate;
incubating the second substrate or portion thereof with the at least one control sample in an extraction solvent so as to produce a supernatant for the at least one control sample that comprises the at least one target molecule and the internal standard molecules;
detecting the at least one target molecule and the internal standard molecules in the control sample supernatant;
generating a calibration curve based on the detection of the at least one target molecule and the internal standard molecules in the control sample supernatant; and
quantifying the amount of the at least one target molecule in the test sample based on the amount of the at least one target molecule and the amount of the internal standard molecules detected in the test sample supernatant and correlation with the calibration curve.

11. The method of claim 1, wherein the fluid test sample is selected from biofluid such as excreted fluid (such as urine or sweat), secreted fluid (such as saliva, tears, breast milk or bile), fluid obtained from within a subject (such as blood, plasma, serum or cerebrospinal fluid), or fluid generated as a result of a pathological process (such as blister or cyst fluid), food sample, plant sample, environmental sample, or the like.

12. The method of claim 1, wherein incubating comprises placing the substrate or portion thereof in a container, and sonicating the substrate in the presence of the extraction solvent, and/or wherein detecting comprises performing mass spectrometry analysis, and wherein the extraction solvent optionally comprises a C1-3 linear or branched chain monoalcohol, in particular methanol.

13. (canceled)

14. The method of claim 1, wherein the target molecule is selected from peptides, proteins, heavy metals, toxins, drugs and their metabolites, amino acids, free carnitine, acylcarnitines and any combinations thereof in a fluid test sample, or, optionally, wherein the target molecule is as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, citrulline, ornithine, free carnitines, acetyl carnitine, propionylcarnitine, butyrylcarnitine, isovalerylcarnitine, glutarylcarnitine, hexanoylcarnitine, octanoylcarnitine, decanoylcarnitine, lauroylcarnitine, myristoylcarnitine, palmitoylcarnitine, and/or stearoylcarnitine, and/or any combinations thereof, wherein, optionally, the target molecule is phenylalanine.

15. (canceled)

16. The method of claim 2, wherein the stable isotope labeled internal standard molecule is selected from stable isotope labeled amino acids, free carnitine, acylcarnitines, and any combinations thereof, such as ²H4-alanine, ¹³C6-,¹⁵N4-arginine, ¹³C4-,¹⁵N2-asparagine, ²H3-aspartic acid, ¹³C3-cysteine, ²H3-glutamic acid, ¹³C5-,¹⁵N2-glutamine, ¹³C-,¹⁵N-glycine, ¹³C6-,¹⁵N3-histidine, ¹³C6-,¹⁵N-isoleucine, ²H3-leucine, ¹³C6-,¹⁵N2-lysine, ²H3-methionine, ¹³C6-phenylalanine, ¹³C5-,¹⁵N-proline, ¹³C3-serine, ¹³C4-threonine, ¹³C11-,¹⁵N2-tryptophan, ¹³C6-tyrosine, ²H8-valine, ²H2-citrulline, ²H6-ornithine, ²H9-carnitine, ²H3-acetylcarnitine, ²H3-propionylcarnitine, ²H3-butyrylcarnitine, ²H9-isovalerylcarnitine, ²H3-glutarylcarnitine, ²H3-hexanoylcarnitine, ²H3-octanoylcarnitine, ²H3-decanoylcarnitine, ²H3-lauroylcarnitine, ²H9-myristoylcarnitine, ²H3-palmitoylcarnitine, and/or ²H3-stearoylcarnitine, and/or any combinations thereof, wherein optionally, the stable isotope labeled internal standard molecule is ¹³C6-phenylalanine.

17. (canceled)

18. The method of claim 1, wherein the at least one target molecule is associated with a metabolic disorder, the metabolic disorder optionally comprising a newborn metabolic disorder, wherein, optionally, wherein the metabolic disorder is phenylketonuria, CACT deficiency or primary carnitine deficiency.

19. (canceled)

20. The method of claim 1, wherein the predetermined volume is about 1 to about 100 microliters, or about 5 to about 15 microliters, or about 35 to about 65 microliters, or about 10 microliters, or about 50 microliters.

21. A microfluidic device comprising:
an inlet for application of a fluid test sample;
a metering channel arranged in fluid communication with the inlet for receiving at least a portion of the fluid test sample from the inlet, the metering channel having a predetermined volume; and
a substrate arranged to receive a metered volume of the fluid test sample from the metering channel, the substrate comprising a predetermined amount of internal standard molecules.

22. The microfluidic device of claim 21, wherein the substrate is comprised of a liquid-absorbing material, wherein the substrate is a disc comprising a diameter of about 5-7 mm and a thick about 1-2 mm.

23. (canceled)

24. (canceled)

25. The microfluidic device of claim 21, wherein the internal standard molecules optionally comprise a label, the label optionally being a stable isotope selected from the group consisting of ²H, ¹³C, ¹⁵N, ¹⁸O, ³⁴S, or any combinations thereof, and wherein the stable isotope labeled internal standard molecule is, optionally, in an amount range of 0.1-5 mmol.

26. (canceled)

27. (canceled)

28. (canceled)

29. The microfluidic device of claim 25, wherein the stable isotope labeled internal standard molecule is selected from stable isotope labeled amino acids, free carnitine, acylcarnitines, and any combinations thereof, such as ²H4-alanine, ¹³C6-,¹⁵N4-arginine, ¹³C4-,¹⁵N2-asparagine, ²H3-aspartic acid, ¹³C3-cysteine, ²H3-glutamic acid, ¹³C5-,¹⁵N2-glutamine, ¹³C-,¹⁵N-glycine, ¹³C6-,¹⁵N3-histidine, ¹³C6-,¹⁵N-isoleucine, ²H3-leucine, ¹³C6-,¹⁵N2-lysine, ²H3-methionine, ¹³C6-phenylalanine, ¹³C5-,¹⁵N-proline, ¹³C3-serine, ¹³C4-threonine, ¹³C11-,¹⁵N2-tryptophan, ¹³C6-tyrosine, ²H8-valine, ²H2-citrulline, ²H6-ornithine, ²H9-carnitine, ²H3-acetylcarnitine, ²H3-propionylcarnitine, ²H3-butyrylcarnitine, ²H9-isovalerylcarnitine, ²H3-glutarylcarnitine, ²H3-hexanoylcarnitine, ²H3-octanoylcarnitine, ²H3-decanoylcarnitine, ²H3-lauroylcarnitine, ²H9-myristoylcarnitine, ²H3-palmitoylcarnitine, and/or ²H3-stearoylcarnitine, and/or any combinations thereof.

30. (canceled)

31. A screening kit for determining a quantity of at least one target molecule in a test sample by using internal standard molecules, the kit comprising:
at least one substrate, and
at least one of stable isotope labeled internal standard molecules, which can be a concentrated liquid or dried powder form; or
at least one substrate pre-loaded with at least one of stable isotope labeled internal standard molecules of a known amount,
wherein the kit optionally comprises at least one extraction solvent, and optionally comprises a loading means for loading the at least one substrate with at least one of stable isotope labeled internal standard molecules.

32. (canceled)

33. (canceled)
